(19) United States
(12) Patent Application Publication
(10) Pub. No.: US 2012/0134918 A1
(43) Pub. Date: May 31, 2012

(54) GUM ARABIC COATED 198GOLD RADIOACTIVE NANOPARTICLES FOR CANCER THERAPY

(75) Inventors: Kattesh V. Katti, Columbia, MO (US); Raghuraman Kannan, Columbia, MO (US); Cathy S. Cutler, Columbia, MO (US)

(73) Assignee: The Curators of the University of Missouri, Columbia, MO (US)

(21) Appl. No.: 13/293,827

(22) Filed: Nov. 10, 2011

Related U.S. Application Data
(60) Provisional application No. 61/456,808, filed on Nov. 12, 2010.

(51) Int. Cl. A61K 51/12; A61P 35/00; A61M 36/06; B82Y 15/00; B82Y 5/00

(52) U.S. Cl. 424/1.29; 427/5; 977/773; 977/915; 977/927

(57) ABSTRACT

The invention provides a cancer therapeutic and imaging agent comprising a solution containing Gum Arabic coated ¹⁹⁸Au nanoparticles. The Gum Arabic coated ¹⁹⁸Au nanoparticles have been demonstrated experimentally shown to have a surprising efficacy for a single dose direct injection, reducing tumors in analog mice by 82% over a short period of time. The particles of the invention have a believed optimal size for therapy and imaging applications, and can be used as a theranostic agent in the treatment of needle accessible cancers. The invention also provides a method for forming Gum Arabic coated ¹⁹⁸Au nanoparticles. A gold foil is irradiated to produce ¹⁹⁸Au foil. The foil is dissolved to form radioactive gold salt. The salt is dried, and then reconstituted to form a ¹⁹⁸Au nanoparticle precursor. The precursor is reduced with a reducing agent in an aqueous solution including Gum Arabic to form Gum Arabic coated ¹⁹⁸Au nanoparticles.

### PRIORITY CLAIM AND REFERENCE TO RELATED APPLICATION

The application claims priority under 35 U.S.C. §119 from prior provisional application Ser. No. 61/456,808, which was filed Nov. 12, 2010.

### STATEMENT OF GOVERNMENT INTEREST

The invention was made with government support under Grants No. CA119142, CA128460 and HHSN261200800065C from the National Institute for Health. The US Government has certain rights in the invention.

### FIELD

A field of the invention is cancer therapy agents and therapeutic methods. A specific application of the invention is to prostrate therapy.

### BACKGROUND

Despite widespread attention, testing, treatments and therapies, prostate cancer remains the second most deadly cancer in men, after lung cancer. Improving current therapies could potentially extend the lives of thousands of men that contract the disease.

Currently accepted diagnoses for prostate cancer start with a screening test, either a digital rectal exam, during which a doctor feels the prostate to check for irregularity or enlargement, with a blood test to check the level of prostate-specific antigen (PSA), in the blood.

Another technique for prostate cancer diagnosis is ultrasound imaging. This is complicated by the location of the prostate deep inside the pelvis.

Post-diagnosis treatments include heightened monitoring and observation, radical prostatectomy, external-beam radiation therapy, and brachy therapy. Each of these techniques has some drawbacks, and the shortened life-time and fatality rate for prostate cancer remains unacceptably high. There remains a need for new and improved therapeutic modalities, especially, for treating inoperable prostate cancer.

Typical low dose brachy therapy use radioactive seeds of Iodine-125 (half life of 60 days; photon energy of 27 keV) or Palladium-103 (half-life of 16.99 days electron capture decay with the emission of characteristic X-rays at 20-23 keV and Auger electrons) are placed permanently into the prostate gland. These agents deliver a low dose of radiation over a period of several months. Difficulties include the accurate placement of the seeds and ability to accurately control dosage.

Gold-198, because of its higher energy of emission (βmax=0.96, MeV; half-life of 2.7 days) has been used as a permanent implant either alone or as an adjunct to external-beam radiation therapy of cancers. See, e.g., Knight P J, et al., “The use of Interstitial Radiation Therapy in the Treatment of Persistent, Localized, and Unresectable Cancer in Children,” Cancer 57: 951-4 (1986); Rich T. A., “Radiation Therapy for Pancreatic Cancer: Eleven Year Experience at the JCRT,” Int J Radiat Oncol Biol Phys. 11:759-63 (1985). Brachytherapy implants of large radioactive gold seeds provide rapid delivery of radiation at a very high dose rate, thus avoiding some of the radiologic problems associated with iodine. However, because of the high heterogeneity of radioactive ¹⁹⁸Au seeds and liquids, oncologists have developed a consensus that a majority of patients receiving low/high energy brachy therapy will experience post treatment symptoms including adverse side effects to severe clinical complications. Recognized complications include proctitis, cystitis, incontinence and rectal bleeding. See, e.g., Dall'Era M A, et al, “Hyperbaric Oxygen Therapy for Radiation Induced Proctopathy in Men Treated for Prostate Cancer,” J Urol 176: 87-90 (2006).

Non-radioactive nanoparticles have been investigated for target specificity and increased retention for significant improvement in the treatment of the prostate and various inoperable tumors. Gommersall L, et al., “Nanotechnology in the Management of Prostate Cancer,” BJU Int 102: 1493-5 (2008); Nie S, et al., “Nanotechnology Applications in Cancer,” Annu Rev Biomed Eng 9: 257-88 (2007). These studies have shown that non-radioactive nanoparticles can aid in detection of prostate specific antigen in blood samples, the use of single walled carbon nanotubes for laser based treatments of cancer, and liposomal delivery of drugs to cancer sites.

Therapy applications of gold based non-emitting nanoparticles have been studied. Katti K K, et al., “In Vitro and in Vivo Antitumor Properties of Tetrakis((Trishydroxy-Methyl)Phosphine)Gold(I) Chloride,” J Med Chem 46: 1130-2 (2003). Gold nanoparticles have an affinity to leaky tumor vasculature which are manifested in angiogenesis of tumor growth. Mukherjee P, et al., “Antiangiogenic Properties of Gold Nanoparticles,”. Clin Cancer Res 11: 3530-4 (2005). Imaging applications of gold based nanoparticles have also been separately studied. Production of tumor specific imaging agents is pursued through the non-radioactive tumor specific gold nanoparticles for diagnostic applications. Kannan R, et al., “Nanocompatible Chemistry Toward Fabrication of Target-Specific Gold Nanoparticles,” J Am Chem Soc 128: 11342-3 (2006)

Gold nanoparticles in X-ray CT imaging and cancer therapy have been studied as a superior replacement for iodine X-ray contrast agents. Hainfeld J F, et al., “Gold Nanoparticles: A New X-ray Contrast Agent,”. Br J Radiol 79: 248-53 (2006). Poly{¹⁹⁸Au} radioactive gold/dendrimer composite nanodevices have been fabricated as a candidate for brachytherapy. Khan MK, et al., “Fabrication of {¹⁹⁸Au⁰} Radioactive Composite Nanodevices and their use for Nanobrachytherapy,” Nanomedicine 4: 57-69 (CND) (2008). Khan et al. studied poly{¹⁹⁸Au} radioactive gold/dendrimer composites sized between 10 nm and 29 nm and their utility in targeted radiopharmaceutical dose delivery to tumors. They reported that single intratumoral injection composites in PBS (phosphate buffered solution) delivered a dose of 74 micro Ci after eight days and resulted in a statistically significant 45% reduction in tumor volume, compared to untreated groups and those injected with the “cold” nanodevice. There are synthetic difficulties of fabricating dendrimers, and the synthesis of ¹⁹⁸Au-dendrimer nanoparticles is in a nuclear reactor by irradiation. This makes fabrication expensive and complex.

The currently used brachy therapy agents including, iodine-125 or palladium-103 radioactive seeds and, Y-90 immobilized glass microspheres (Therasphere™) utilize agents of 20-100 microns in size to achieve selective internal radiation therapy (SIRT). The limited natural affinity of these microspheres toward tumor vasculature coupled with significantly larger 50-100 micron sizes of brachy seeds as compared to the porosity of tumor vasculature (150-300 nm) results in limited retention and significant leakage of therapeutic dose away from the tumor site. Such clinical problems have resulted in decreased efficacy, acute toxic side effects and lower tumoricidal activity of brachy therapy agents.

### SUMMARY OF INVENTION

The invention provides a cancer therapeutic and imaging agent comprising a solution containing Gum Arabic coated ¹⁹⁸Au nanoparticles. The Gum Arabic coated ¹⁹⁸Au nanoparticles have been demonstrated experimentally shown to have a surprising efficacy for a single dose direct injection, reducing tumors in analog mice by 82% over a short period of time. The particles of the invention have a believed optimal size for therapy and imaging applications, and can be used as a theranostic agent in the treatment of needle accessible cancers. The invention also provides a method for forming Gum Arabic coated ¹⁹⁸Au nanoparticles. A gold foil is irradiated to produce ¹⁹⁸Au foil. The foil is dissolved to form radioactive gold salt. The salt is dried, and then reconstituted to form a ¹⁹⁸Au nanoparticle precursor. The precursor is reduced with a reducing agent in an aqueous solution including Gum Arabic to form Gum Arabic coated ¹⁹⁸Au nanoparticles.

### BRIEF DESCRIPTION OF DRAWINGS

FIG. 1 illustrates therapeutic data for tumor bearing control mice and tumor bearing mice treated with Gum Arabic radioactive ¹⁹⁸Au nanoparticles; and

FIGS. 2A-2C illustrate blood count data for mice in the therapeutic studies that were conducted.

### DETAILED DESCRIPTION OF INVENTION

The invention provides biocompatible Gum Arabic coated radioactive ¹⁹⁸Au nanoparticles as new generation of therapeutic agent for the treatment of cancer. A preferred cancer therapy is prostate cancer therapy. In addition, Gum Arabic coated radioactive ¹⁹⁸Au nanoparticles of the invention can serve as imaging agents, thereby acting as theranostic agent for dual imaging and therapy purposes.

The GA ¹⁹⁸Au nanoparticles, can be synthesized under clinical settings using ¹⁹⁸Au produced at a reactor. This provides a simpler fabrication than is required for in-reactor fabrication.

Specific non-emitting nanoparticles in the above studies have individually acted as imaging or therapy agents. However, singular agents with dual imaging and therapeutic (“theranostic”) capabilities of nanoparticles have not be provided to the inventors' knowledge. The present inventors have recognized that a dual theranostic agent would provide tremendous consistency in the follow up of therapy studies and could also minimize regulatory steps leading to final approval by the Food and Drug Administration (FDA). The inventors have also recognized that theranostic properties of gold offer realistic clinical possibilities for use as dual imaging and therapy agents.

Preferred Gum Arabic coated radioactive ¹⁹⁸Au nanoparticles of the invention have demonstrated surprising therapeutic efficacy in human model severe combined immune deficiency (SCID) mice bearing human prostate tumor xenografts. Experiments demonstrated clinically significant tumor regression and effective control in the growth of prostate tumors over 30 days after intratumoral administration of a single dose of β-emitting Gum Arabic-¹⁹⁸Au nanoparticles (70 Gy). Three weeks after administration of Gum Arabic coated ¹⁹⁸Au nanoparticles, tumor volumes for the treated animals were five-fold smaller as compared to control groups and the overall reduction in tumor volume reached an unprecedented 82% at the end of treatment.

The efficacy demonstrated in the testing mentioned above provides demonstrated utility in treating various forms of human cancers including any cancer that is accessible by direct injection. An example important cancer that is expected to be injection accessible is prostate cancer. Without being bound to any theory and without any theory being necessary to enable the invention, it is believed that the size of preferred embodiment radioactive gold nanoparticles is perhaps optimal for targeting individual tumor cells and to penetrate through tumor vasculature and porosity. A typical Gum Arabic coated ¹⁹⁸Au nanoparticle of a preferred embodiment has a core in the range of ˜12-18 nm and a ˜85 nm hydrodynamic diameter.

Preferred embodiment Gum Arabic coated radioactive ¹⁹⁸Au nanoparticles were made and tested as a therapeutic agent. The experiments will be discussed and illustrated and artisans will understand broader aspects of the invention from the discussed preferred embodiments, experiments, and studies.

Gum Arabic Radioactive ¹⁹⁸Au Nanoparticles and Non-Emitting Analog Synthesis

University of Missouri Research Reactor (MURR) irradiation facilities were used for the production of ¹⁹⁸Au. Gold foil 0.5 mg was irradiated at a thermal flux of 8×10¹³n/cm²/s and an epithermal flux of 6×10¹² n/cm²/s to form a radioactive ¹⁹⁸Au foil. The radioactive foil was dissolved into radioactive ¹⁹⁸Au salt with aqua regia and dried down to near dryness and then reconstituted into a radioactive ¹⁹⁸Au nanoparticle precursor with 50 μL of 0.05 N HCl to form radioactive H¹⁹⁸AuCl₄. The radioactive precursor H¹⁹⁸AuCl₄ in 0.05N HCl was added to aqueous solutions of a gum Arabic stabilizing agent followed by the addition of a reducing agent, trimeric alanine conjugate, (P(CH₂NHCH(CH₃)COOH)₃) (“tris-hydroxymethyl phosphine alanine (THPAL)”). A color change from yellow to a red-purple was observed to yield nanoparticles stabilized by gum Arabic. The solution was characterized by UV-VIS spectrophotometry which showed a plasmon absorption band around 540 nm for ¹⁹⁸Au nanoparticles, characteristic of nanoparticulate ¹⁹⁸Au formation. The solution was made isotonic and brought of neutral pH by addition of NaOH and phosphate buffered saline. This plasmon transition at the tracer level for ¹⁹⁸Au nanoparticle was further confirmed by measurement using an Ocean Optics USB 2000 UV-Visible spectrophotometer.

The synthesis produced Gum Arabic radioactive ¹⁹⁸Au nanoparticles with over 98% yields. Absorption measurements indicate that the plasmon resonance wavelength, λmax, and plasmon line width, Δλ, of Gum Arabic conjugated ¹⁹⁸Au nanoparticless (Gum Arabic ¹⁹⁸Au nanoparticles) are ˜540 nm and 151 nm, respectively. The size of the Gum Arabic ¹⁹⁸Au nanoparticles was found to be 12-20 nm as measured from TEM images. However, the hydrodynamic radius as determined by Zeta-potential measurement using Malvern instruments reveals that 95% of Gum Arabic ¹⁹⁸Au nanoparticles conjugates have an average diameter of 85 nm. It may be noted that the TEM measurements have revealed that the average diameter of the metallic part of the nanoparticles to be of 15-20 nm in size. This result implies that the Gum Arabic coating over ¹⁹⁸Au nanoparticles occupies ˜50-80 nm in thickness.

Gum Arabic non-emitting gold nanoparticles were synthesized from HAuCl₄ using similar protocols for synthesizing ¹⁹⁸Au nanoparticles and used for establishing the stability and biocompatibility properties of the conjugates prior to in vivo application of the Gum Arabic ¹⁹⁸Au nanoparticles for therapy.

Dynamic light scattering (DLS) method revealed that the hydrodynamic diameter of Gum Arabic ¹⁹⁸Au gold nanoparticles is 85 nm and the Zeta Potential (ζ) is −24.5±1.5 mV.

Stability and Biocompatibility Assessment.

An issue of importance for in vivo applications therapeutic and/or imaging applications of Gum Arabic ¹⁹⁸Au nanoparticles is stability under a variety of in vitro profiles. This permits creation, delivery and storage of a therapeutic agent for use in therapy.

The stability of the Gum Arabic ¹⁹⁸Au nanoparticles was evaluated by monitoring the plasmon (λmax) and plasmon band width (Δλ) in 0.2M Cysteine, 0.2M Histidine, HSA, or 35% NaCl over a 24-48 day periods. The plasmon wavelength and width in all the above formulations shifts ˜10 nm. These data indicate that the ¹⁹⁸Au nanoparticles are intact, and thus demonstrate high in vitro stability of Gum Arabic ¹⁹⁸Au nanoparticles in biological fluids at physiological pH.

Dilution Assessment.

In biomedical applications that require lower concentrations of ¹⁹⁸Au nanoparticles, it is vital that dilution of Gum Arabic ¹⁹⁸ Au nanoparticles solutions does not alter their characteristic chemical and photophysical properties. The effect of dilution on the stability of ¹⁹⁸Au nanoparticles was also tested by monitoring the plasmon resonance wavelength (λmax) and band width (Δλ) after each of a series of successive additions of 0.2 mL of doubly ionized (DI) water to 1 ml of radioactive gold nanoparticles solutions. The absorption intensity at λmax has been found to be linearly dependent on the concentration of radioactive gold nanoparticles, in accordance with Beer Lambert's law. Solutions of Gum Arabic ¹⁹⁸Au nanoparticles did not show a change in the λmax and Δλ of ¹⁹⁸Au nanoparticles at dilutions in the range of 10−6M-10⁻⁸M. These are typical concentrations encountered when working at cellular levels in vivo.

Platelet Aggregation

Platelets, or thrombocytes, are the cell fragments circulating in the blood that are involved in the cellular mechanisms of primary hemostasis leading to the formation of blood clots. Platelet-rich plasma obtained from fresh pooled human whole blood and incubated with control or test sample for 15 min at 37° C. The platelet-rich plasma was analyzed using a Z2 particle counter and size analyzer to determine the number of active platelets. Percent aggregation was calculated by comparing the number of active platelets in the test sample to the number in the control baseline tube. In the ideal situation, the nanoparticles should neither lead to nor inhibit platelet aggregation.

The results tests demonstrated that Gum Arabic ¹⁹⁸Au nanoparticles did not result in any platelet aggregation. It is important to recognize that Gum Arabic ¹⁹⁸Au nanoparticles displayed inhibition for platelet aggregation as demonstrated by results from our detailed studies which involved cocktail mixtures of Gum Arabic ¹⁹⁸Au nanoparticles, positive control and negative controls.

Hemolysis

Hemolysis studies assessed the biocompatibility of nanoparticles in terms of their hemolytic properties upon direct contact with blood. The test procedure is run under static conditions. The amount of hemoglobin released from erythrocytes in plasma by the test sample is measured spectrophotometrically at 540 nm concurrently with the negative and positive controls. The method involves directly exposing the test nanoparticle sample to a blood cell suspension for 3 hr at 37° C. The extract method involves exposing nanoparticles to the blood cell suspension for 3 hr at 37° C. After exposure, the blood suspensions are centrifuged to separate the free hemoglobin (produced by lysed cells) from the unlysed cells. If the test nanoparticles also exhibit absorbance close to 540 nm (as in the case of the gold nanoparticles) the supernatant is further centrifuged at high speed to remove the nanoparticles. The supernatant is then combined with a cyanide-based reagent to convert the multiple types of hemoglobin released to a common cyanide based form. The cyanide-based form (cyanmethemoglobin) can be read spectrophotometrically as a single peak at 540 nm. A hemolytic index (percent hemolysis) is determined using the optical density readings of the sample and data obtained from a hemoglobin standard curve and the hemolytic index is used to evaluate the acute hemolytic properties of nanoparticles. The results demonstrated that Gum Arabic ¹⁹⁸Au nanoparticles did not result in any hemolysis.

Animal Model Studies

Animal studies were approved by the Institutional Animal Care and Use Committees of the Harry S. Truman Memorial Veterans Hospital and the University of Missouri, and were performed in accordance with the Guide for the Care and Use of Laboratory Animals.

Therapeutic Efficacy and Pharmacokentic

Human prostate cancer cell line PC-3 was obtained from the American Type Culture Collection (ATCC; Manassas, Va.), and cultured according to ATCC recommendations by the University of Missouri Cell and Immunobiology Core facility. Mice received ear tag identifiers under inhalational anesthesia (isoflurane/oxygen) followed by unilateral, subcutaneous hind flank inoculations of 10×10⁶ PC-3 cells suspended in 0.1 mL of sterile Dulbecco's phosphate buffered saline (DPBS) and Matrigel® (2:1, v:v). Solid tumors were allowed to develop over a period of 3 weeks, and animals were randomized.

Detailed therapeutic capabilities of Gum Arabic ¹⁹⁸Au nanoparticles have been evaluated using SCID mice with prostate tumor xenografts as tumor models. Severely compromised immunodeficient (SCID) mice bearing a flank model of human prostate cancer derived from a subcutaneous implant of 10 million PC-3 cells were used for therapeutic efficacy and pharmacokinetic studies. The female ICRSC-M SCID mice (4-5 weeks of age; Taconic Farms, Hudson, N.Y.) were group housed in a temperature and humidity controlled, pathogen-free barrier facility. Animals were maintained on a 12 h light-dark cycle, and had access to sterilized standard chow and water ad libitum. Animals were allowed to acclimate for 7-10 days prior to initiation of work.

A retention in vivo study involved intratumoral administration of Gum Arabic ¹⁹⁸Au nanoparticles (1.5 μCi/tumor) to groups (n=5) of SCID mice bearing human prostate cancer xenografts had shown retention of over 71% of the injected dose within the tumor xenografts at 30 minutes that declined to 35% by 24 hours. For a therapy study, unilateral solid tumors were allowed to grow three weeks, and animals were randomized (denoted Day 0) into control and treatment groups (n=7) with no significant differences in tumor volume. On Day 8, 30 μL of Gum Arabic ¹⁹⁸Au nanoparticles (408 μCi) was injected directly into the tumor to deliver an estimated dose of 70 Gy.

Control animals received 30 μL Dulbecco's PBS. Tumors were then measured twice each week.

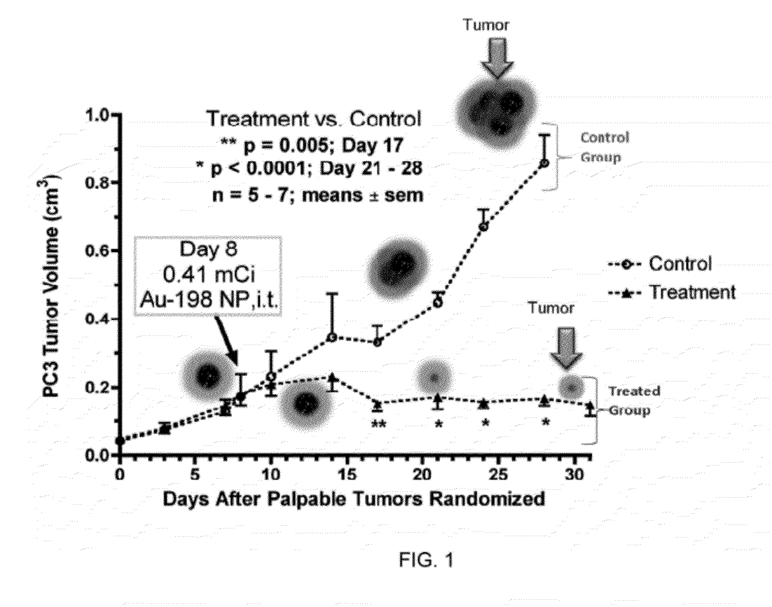

The therapeutic efficacy of Gum Arabic ¹⁹⁸Au nanoparticles for radiotherapy in PC-3 (human prostate) bearing mice. FIG. 1 shows the surprising and excellent results from the single-dose radiotherapy study of Gum Arabic ¹⁹⁸Au nanoparticles in SCID mice bearing xenografts of human prostate cancer derived from the PC-3 cell line. As seen in FIG. 1, tumor volumes for the treated animals were five-fold smaller after 31 days as compared to control groups and the overall reduction in tumor volume reached a level of 82% at the end of treatment.

On Day 0, animals were randomized into two groups of seven showing no differences (p>0.05) in tumor volume or body weight. Eight days later, one group received 408 μCi of ¹⁹⁸Au nanoparticles that was injected directly into the tumor to deliver an estimated dose of 70 Gy. The control group received only the buffered saline vehicle. Within one week (Day 14), tumor growth in the treated animals appeared to be slowing with respect to controls. Nine days after Gum Arabic ¹⁹⁸Au nanoparticles administration (Day 17), tumor volumes were two-fold lower (p<0.005) for treated animals compared to controls. This significant therapeutic effect was maintained throughout the 30 day long study. Three weeks after Gum Arabic ¹⁹⁸Au nanoparticles administration, on Day 28 from randomization, tumor volumes for the control animals were fully five-fold greater with respect to those for the radiotherapy group (p<0.0001; 0.86±0.08 vs. 0.17±0.02 cm³. On Days 16 and 26, one animal from the control group had to be euthanized due to excessive weight loss (>20%). On Day 28, the five animals remaining in the control group were euthanized due to continued weight loss, deteriorating overall health status, and risk of tumor ulceration. By contrast, none of the seven animals in the treatment group reached early termination criteria. They did exhibit a transient weight loss that peaked at −17.6±2.4% on Day 17, but recovered to −10.6±2.9% by Day 31. This results demonstrate surprising efficacy and an unexpected level of tumor reduction.

With reference to FIG. 1, (Day 0) into control and treatment groups (n=7) having no significant difference in tumor volumes (probability p=0.64) or body weights (p=0.17). Tumor volumes were estimated from caliper measurements using the formula V=length×width×depth. The mean estimated tumor mass for each group was 202 mg. On Day 8, animals in the treatment group received intratumoral (i.t.) injections of Gum Arabic ¹⁹⁸Au nanoparticles (408 μCi) in DPBS (30 μL) while under inhalational anesthesia. Similarly, control animals received 30 μL, of DPBS i.t. No significant difference (p=0.93) in tumor volume or body weight (p=0.21) was noted between the groups at the time of injection. Tumor volumes, body weights and health status were then determined twice each week.

At the end of the study (Day 31), mice were euthanized by cervical dislocation, and blood samples were collected by cardiac puncture. Samples of tumor, liver and other organs of interest from the treatment group were also harvested, weighed and counted for radioactivity in comparison to a sample of the injected dose using an automated γ-counter.

The control group of SCID mice (n=7) received no experimental manipulations, and were maintained through the end of study for determination of normal blood cell and platelet counts for comparison to the control and treatment groups. Complete blood counts (CBC) were determined by the University of Missouri Research Animal Diagnostic Laboratory (RADIL) using an Abbott Cell-Dyn® 3500 analyzer on blood samples obtained by cardiac puncture and treated with tripotassium ethylenediaminetetraacetate (K₃-EDTA; Vacuette Mini-Collect®).

Tumors harvested from the treatment group consisted largely of necrotic tissue, indicating extensive tumor cell kill. These residual tumor tissue samples still contained 19.9±4.2% of the injected dose (ID) of Gum Arabic ¹⁹⁸Au nanoparticles. Liver contained 0.91±0.26% ID, kidney 0.13±0.01% ID and small intestines 0.09±0.00% ID. Levels of radioactivity noted for blood, heart, lung, spleen, stomach and pancreas were barely distinguishable from background, while remaining carcass contained 18.5±4.6% ID. The observed insignificant or no radioactivity in liver, intestine and various non-target organs unequivocally established that the therapeutic payload resided within the tumor site throughout the 30 day long treatment regimen.

The therapeutic efficacy studies of Gum Arabic ¹⁹⁸Au nanoparticles have demonstrated excellent retention of Au therapeutic dose within the prostate tumor site in the mice. At the end of the 30 day study, 20% of the injected radioactivity remained in the tumor, over 20-fold greater than that noted for the liver, the organ having the next highest concentration of Gum Arabic-radioactive gold nanoparticles. The Gum Arabic ¹⁹⁸Au nanoparticles therapy in vivo was well tolerated. The treatment group showed only transient weight loss with no early terminations. This stands in sharp contrast to the control group that showed continued weight loss and deterioration of health status leading to early loss of two animals. The measurements of white and red blood cells, platelets, and lymphocytes, within the treatment group resembled that of the normal SCID mice. This observation provides further evidence on the therapeutic efficacy, and concomitant in vivo tolerance and non-toxic features of Gum Arabic ¹⁹⁸Au nanoparticles.

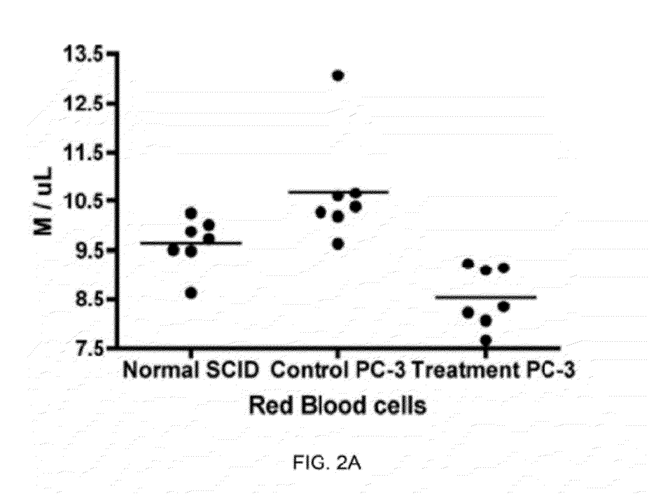

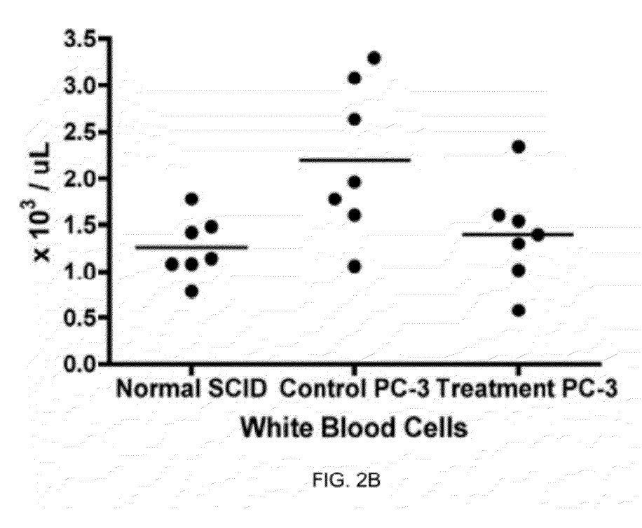

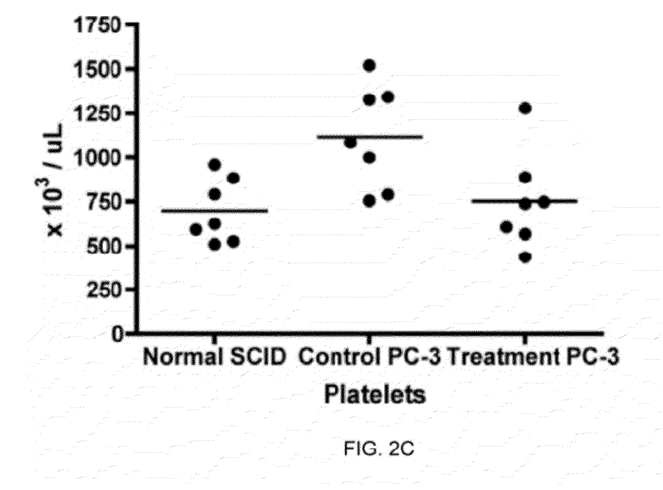

Blood parameters were compared between the treatment and control groups with baseline levels obtained from a third group of SCID mice that received no manipulations. Analysis of variance followed by a post hoc Dunnett's test was employed. The mean white blood cell (WBC) count for Gum Arabic ¹⁹⁸Au nanoparticles treated animals (1.40±0.21×10³/μL) was not significantly (p>0.05) different from the baseline WBC (1.25±0.13×10³/μL) of the normal SCID mice. By contrast, the WBC for the untreated tumor-bearing group (2.20±0.31×10₃/μL) was significantly (p<0.05) elevated by 75% with respect to baseline measures. Red blood cell counts between the three groups varied only slightly, by approximately 10%, and the hematocrit was not different between the three groups, as seen in FIGS. 2A-2C. Platelet levels were elevated by 60% from baseline for the untreated tumor bearing animals (1118±111×10³/μL vs. 698±68×10³/μL; p<0.05), while those from the radiotherapy group (752±104×10³/μL) showed no significant difference (p>0.05).

Imaging Studies

Male NCR nu/mice were obtained from Taconic. The mice were 7-8 weeks old on Day 1 of the experiment. All animals weighed >19.5 g at the initiation of experiment. The imaging studies used a non-radioactive particle coated with Gum Arabic and sized like the ¹⁹⁸Au nanoparticles used in the therapy studies.

All animals were then injected with a fixed volume of 100 μL (8 mg/mL of gold concentration) of Gum Arabic gold nanoparticles. The Gum Arabic gold nanoparticles were administered via direct injection into the tumor. Four injections of 0.2 mL of Gum Arabic gold nanoparticles of 8 mg/mL solutions were administered during the first 24 hr and three injections were given between the 24 and 48 hr time points. Micro-CT scans were performed on all mice, immediately before treatment, then again at 1, 6, 24 and 48 hr post-administration of the treatment. A GE RS150 small animal micro-CT scanner was used for this experiment. Animals that received intratumoral administration of Gum Arabic gold nanoparticles showed a change in tumor contrast compared to pre-treatment images, at all post-treatment CT time points. Contrast, as observed by changes in HU numbers, was observed in the periphery of the tumor, suggesting perforation of Gum Arabic gold nanoparticles to the more viable/hyper-perfused region of the tumor.

Gum Arabic gold nanoparticles in tumor bearing mice resulted in excellent contrasts of tumors at all post-treatment CT time points. The contrast was very bright and presented in a ring pattern that may be indicative of the more peripheral region of the tumor and homogeneous spreading and penetrability of the gold contrast agent. In the tests, Tumor A had nanoparticles and Tumor B had none, and there is a clear contrast enhancement in Tumor A that received a nanoparticle injection. The degree of contrast enhancement generally increased at each imaging time point. The total increase in CT number over the study duration was 13.5%. It is noteworthy that the final 24 hr imaging time point was not immediately preceded by a dose of the Gum Arabic gold nanoparticles, as were the earlier (1, 6, and 12 hr) post-treatment imaging time points. This suggests that contrast enhancement was dominated by Gum Arabic gold nanoparticles retained in the tumor cells over days.

Theranostic Applications

The therapy and imaging results show that the present Gum Arabic ¹⁹⁸Au nanoparticles provide a theranostic agent for the treatment of prostate cancer and various other cancers that are needle accessible for direct injection. The size range that is well-suited to penetrate into the porous tumor vasculature. The sufficiently smaller size of Gum Arabic ¹⁹⁸Au nanoparticles compared to current brachy agents will allow injections of homogeneously dispersed agent directly into needle accessible tumors with consequent easy passage across the endothelium at tumor sites, and thus is effective in targeting to appropriate endocytosed cancer marking epitopes.

The compelling therapy evidence that showed significant tumor reduction and the good imaging evidence demonstrates a dual function theranostic agent for the treatment of various forms of needle accessible human and animal cancers. The evidence also shows that intratumoral injections of Gum Arabic radioactive gold nanoparticles will not interfere with systemic administration of other therapeutic agents. Therefore, even the large tumors can be effectively treated with homogeneously dispersed injectable Gum Arabic ¹⁹⁸Au nanoparticles for therapeutic applications with consequent benefits to patient community. Currently few brachy therapeutic modalities allow both treatment and assessment of therapeutic response from the same agent.

While specific embodiments of the present invention have been shown and described, it should be understood that other modifications, substitutions and alternatives are apparent to one of ordinary skill in the art. Such modifications, substitutions and alternatives can be made without departing from the spirit and scope of the invention, which should be determined from the appended claims.

Various features of the invention are set forth in the appended claims.

1. A cancer therapeutic and imaging agent comprising a solution containing Gum Arabic coated ¹⁹⁸Au nanoparticles.

2. The cancer therapeutic and imaging agent of claim 1, wherein a gold core of said nanoparticles has a diameter in the range of ˜12-18 nm.

3. The cancer therapeutic and imaging agent of claim 2, wherein said nanoparticles have a Gum Arabic coating with a thickness in the range of ˜50-80 nm.

4. A therapeutic method, comprising the injection of a solution of claim 1 directly into a tumor.

5. The method of claim 6, wherein the tumor is a human prostate tumor.

6. A theranostic method, comprising the injection of a solution of claim 1 directly into a tumor followed by contrast imaging of the tumor area.

7. The method of claim 8, wherein the tumor is a human prostate tumor.

8. The cancer therapeutic and imaging agent of claim 1, wherein the solution is isotonic and of neutral pH.

9. The cancer therapeutic and imaging agent of claim 8, wherein the solution includes NaOH, phosphate buffered saline, and doubly ionized water.

10. A method for forming Gum Arabic coated ¹⁹⁸Au nanoparticles, the method comprising:
irradiating gold foil to produce ¹⁹⁸Au foil;
dissolving the ¹⁹⁸Au foil to form radioactive gold salt;
drying the radioactive gold salt;
reconstituting the radioactive gold salt to form a ¹⁹⁸Au nanoparticle precursor;
reducing the ¹⁹⁸Au nanoparticle precursor with a reducing agent in an aqueous solution including Gum Arabic to form Gum Arabic coated ¹⁹⁸Au nanoparticles.

11. The method of claim 10, wherein said reconstituting comprises reacting with HCL to form radioactive H¹⁹⁸AuCl₄ as the gold precursor.

12. The method of claim 10, wherein the reducing agent comprises a trimeric alanine conjugate.

13. The method of claim 10, further comprising buffering the Gum Arabic coated ¹⁹⁸Au nanoparticles.
